SEQUENCE LISTING (6 sequences)

SEQ ID NO: 1 - RNA, 20 nt, synthetic construct
  source 1..20: mol_type = other RNA
ttaaaaatatatagaataag

SEQ ID NO: 2 - RNA, 20 nt, synthetic construct
  source 1..20: mol_type = other RNA
atgttcattatgttttctct

SEQ ID NO: 3 - RNA, 20 nt, synthetic construct
  source 1..20: mol_type = other RNA
aaagggatactaaaggaaaa

SEQ ID NO: 4 - RNA, 20 nt, synthetic construct
  source 1..20: mol_type = other RNA
agaatatataaagaacttct

SEQ ID NO: 5 - DNA, 105 bp, Homo sapiens
  source 1..105: mol_type = other DNA
ttctgtagtcttttgggttataagcaattcgattcagttcctcttattctatatatttttaaactttgtgtgatactctttttaaatgaagtttttaaaatgttc

SEQ ID NO: 6 - RNA, 20 nt, synthetic construct
  source 1..20: mol_type = other RNA
atatatagaataagaggaac

(19) United States
(12) Patent Application Publication
(10) Pub. No.: US 2023/0340463 A1
(43) Pub. Date: Oct. 26, 2023

(54) Method and Reagents for Reprogramming Endothelial Cells

(71) Applicant: University of Pittsburgh - Of the Commonwealth System of Higher Education, Pittsburgh, PA (US)

(72) Inventor: Stephen Yu-Wah Chan, Pittsburgh, PA (US)

(21) Appl. No.: 18/306,511

(22) Filed: Apr. 25, 2023

Related U.S. Application Data
(60) Provisional application No. 63/334,377, filed on Apr. 25, 2022.

(51) Int. Cl. C12N 15/11; C12N 9/22; C12N 5/074; A61P 9/12

(52) U.S. Cl. CPC C12N 15/11; C12N 9/22; C12N 5/0696; A61P 9/12; C12Q 1/686

(57) ABSTRACT

Provided herein are methods of treating a disease or condition in a patient, such as pulmonary hypertension, having one or more Gs at SNV rs73184087, comprising editing one or both G's at rs73184087 in the patient. Provided herein also are methods of treating a disease or condition in a patient, having one or more As, Ts or Gs at SNV rs73184087, comprising substituting one or both As, Ts or Gs at rs73184087 in the patient with a G. Also provided herein is an iPSC cell or a cell differentiated from the iPSC cell, homozygous for G at SNV rs73184087, having use in screening drugs for their ability to treat a hypoxia-related or ischemia-related disease or condition in a patient, such as pulmonary hypertension.

### CROSS REFERENCE TO RELATED APPLICATIONS

This application claims priority to U.S. Provisional Patent Application No. 63/334,377 filed Apr. 25, 2022, the disclosure of which is incorporated by reference in its entirety.

### STATEMENT REGARDING FEDERAL FUNDING

This invention was made with government support under Grant Nos. HL124021 and HL122596 awarded by the National Institutes of Health. The government has certain rights in the invention.

The Sequence Listing associated with this application is filed in electronic format via Patent Center and is hereby incorporated by reference into the specification in its entirety. The name of the XML file containing the Sequence Listing is 2303065.xml. The size of the XML file is 6,430 bytes and the XML file was created on Apr. 24, 2023.

Chronic lung diseases, such as chronic obstructive pulmonary disease, cystic fibrosis, and bronchopulmonary dysplasia, can result in diffuse chronic alveolar hypoxia and can lead to the development of pulmonary hypertension, such as pulmonary arterial hypertension. Other conditions resulting in hypoxic conditions, or hypoxia-induced conditions include ischemic conditions, such as myocardial infarct, ischemia, ischemia/reperfusion injury, valvular heart disease, congestive heart failure, stroke, thrombus, embolism, peripheral arterial disease, mesenteric ischemia, chronic limb ischemia, and disease-related ischemia, such as with sepsis, cancer, and neurodegeneration. Cellular reprogramming by hypoxia relies upon incompletely defined genomic, epigenetic, and metabolic circuitry. Such fundamental biologic concepts of hypoxia are important for pulmonary hypertension (PH) and its more severe form, pulmonary arterial hypertension (PAH)—diseases of lung blood vessels linked to hypoxia and its master transcription factors HIF-α. HIF-2α in pulmonary arterial endothelial cells (PAECs) is particularly important in promoting this disease. However, the broad heterogeneity of disease- and hypoxia-dependent molecular circuitry has bred confusion regarding the development of crucial endothelial pathophenotypes.

An integrated understanding of genomic, epigenetic, and metabolic landscapes in the hypoxic endothelium and the pulmonary vasculature is lacking. HIF-dependent pathways primarily regulate metabolic and mitochondrial programs known to be dysregulated in hypoxic and diseased pulmonary vasculature in PH. Genome-wide molecular profiling in PH has revealed that epigenetic marks of the genome and associated histones are altered in hypoxia across various PH subtypes. Histone H3 lysine 4 trimethylation (H3K4me3) is often enriched near promoters of activated genes and drives transcription. Although H3K4me3 is increased in hypoxia and controlled by HIF-1α and HIF-2α, the roles of H3K4me3 in orchestrating metabolic reprogramming in hypoxia and PH are still unknown. Moreover, because hypoxia and HIF-2α constitute crucial triggers of World Symposium on Pulmonary Hypertension (WSPH) Group 1 PH (PAH) and Group 3 PH (PH due to hypoxic lung disease), hypoxic regulation of H3K4me3 may exert control over key pathogenic pathways in these multiple PH subtypes. Finally, genomic insights are advancing, in regard to various levels of association between specific genetic variants with PAH risk, survival, and disease severity. However, due to the limited global number of PAH patients, barriers exist in generating a comprehensive catalog of genomic variants causatively linked to disease initiation or progression.

Methods of modulating activity of the HIF-2α pathway, e.g. in a hypoxia-induced condition or hypoxic tissue, or in a condition where increased downstream activity is desired, such as with PH, PAH, myocardial infarct, stroke, disease-related ischemia, embolism, or thrombus, among other conditions such as sepsis, cancer, and neurodegeneration.

### SUMMARY

According to a first embodiment or aspect of the invention, a method of reducing histone methylation by KMT2E, such as reducing H3K4 methylation in a patient having one or more Gs at rs73184087 is provided. The method comprises deleting the one or more of the Gs at rs73184087 or substituting the one or more Gs at rs73184087 with A, T, or C in a cell, tissue, or organ of the patient. The method may be used for treatment of a condition in the patient having one or more Gs at rs73184087 in which expression of HIF-2α is elevated above normal.

According to a further embodiment or aspect of the invention, a method of increasing histone methylation by KMT2E, such as increasing H3K4 methylation in a patient having one or more As, Ts, or Cs at rs73184087 is provided. The method comprises substituting at least one of the one or more bases selected from A, T, or C at rs73184087 with G in a cell, tissue, or organ of the patient using gene editing. The method may be used for treating a condition in a patient having one or more bases selected from A, T, or C at rs73184087 in which expression of HIF-2α is reduced below normal, comprising substituting at least one of the one or more bases selected from A, T, or C at rs73184087 with G in a cell, tissue, or organ of the patient using gene editing.

The following numbered clauses outline various aspects or embodiments of the present invention.

Clause 1. A method of reducing histone methylation by KMT2E, such as reducing H3K4 methylation in a patient having one or more Gs at rs73184087, comprising deleting the one or more of the Gs at rs73184087 or substituting the one or more Gs at rs73184087 with A, T, or C in a cell, tissue, or organ of the patient.

Clause 2. The method of clause 1 for of treating a condition in a patient having one or more Gs at rs73184087 in which expression of HIF-2α is elevated above normal.

Clause 3. The method of clause 1 or 2, wherein the condition is a hypoxia-induced condition or hypoxic tissue in the patient.

Clause 4. The method of any one of clauses 1-3, wherein the patient is homozygous for G at rs73184087, the method comprising substituting both Gs with A, T, or C in a vascular endothelial cell of the patient.

Clause 5. The method of clause 1 or 3, wherein the condition is one of pulmonary hypertension, pulmonary arterial hypertension, myocardial infarct, ischemia/reperfusion injury, ischemia, valvular heart disease, congestive heart failure, stroke, cancer, neurodegeneration, thrombus, embolism, and disease-related ischemia, such as with sepsis—an example of an ischemic condition or ischemia/reperfusion injury.

Clause 6. The method of clause 5, wherein the condition is a myocardial infarct, embolism, or thrombus.

Clause 7. The method of clause 5, wherein the condition is pulmonary hypertension.

Clause 8. The method of clause 5, wherein the condition is pulmonary arterial hypertension.

Clause 9. The method of clause 5, wherein the condition is Von Hippel Lindau disease.

Clause 10. The method of any one of clauses 1-9, wherein the one or more Gs are substituted with A, T, or C using CRISPR/CAS9 editing.

Clause 11. The method of clause 10, wherein the CRISPR/CAS9 editing is performed using a guide RNA (gRNA) target sequence selected from: TTAAAAATATATAGAATAAG (SEQ ID NO: 1) the protospacer adjacent motif (PAM) is AGG; ATGTTCATTATGTTTTCTCT (SEQ ID NO: 2) where the PAM is TGG; AAAGGGATACTAAAGGAAAA (SEQ ID NO: 3) where the PAM is GGG; or AGAATATATAAAGAACTTCT (SEQ ID NO: 4) where the PAM is GGG.

Clause 12. The method of any one of clauses 1-9, wherein the one or more Gs are substituted with A, T, or C using DNA base editing.

Clause 13 The method of clause 12, wherein the one or more Gs at rs73184087 are substituted with A using a cytosine base editor to edit the complementary C to the G at rs73184087 to a T.

Clause 14. The method of any one of clauses 1-9, wherein the one or more Gs are substituted with A, T, or C using prime editing.

Clause 15 The method of any one of clauses 1-14, wherein the one or more Gs at rs73184087 are substituted with A.

Clause 16. A method of increasing histone methylation by KMT2E, such as increasing H3K4 methylation in a patient having one or more As, Ts, or Cs at rs73184087 comprising substituting at least one of the one or more bases selected from A, T, or C at rs73184087 with G in a cell, tissue, or organ of the patient using gene editing.

Clause 17. The method of clause 16, for treating a condition in a patient having one or more bases selected from A, T, or C at rs73184087 in which expression of HIF-2α is reduced below normal, comprising substituting at least one of the one or more bases selected from A, T, or C at rs73184087 with G in a cell, tissue, or organ of the patient using gene editing.

Clause 18. The method of clause 16 or 17, wherein the patient has an anemia, such as anemia in chronic kidney disease; or peripheral vascular disease and limb ischemia, to increase blood supply and angiogenesis; or is in need of ischemic preconditioning and remote ischemic preconditioning.

Clause 19. The method of any one of clauses 16-18, wherein the gene editing is a CRISPR-Cas9 editing, base editing, or prime editing method.

Clause 20. An iPSC homozygous for Gs at rs73184087.

Clause 21. A cell, such as an endothelial cell, differentiated from the iPSC of clause 20.

Clause 22. A method of screening for compounds able to suppress stabilization of KMT2E protein by KMT2E-AS1, comprising culturing under hypoxic conditions a candidate compound with a cell of clause 20 or 21 and determining if expression of KMT2E-AS1 is decreased, for example by reduced lysine trimethylation on histone 3 by reduced activity of H3K4me3 or by direct measurement of KMT2E-AS1 detected, for example by quantitative RT-PCR.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts the surrounding sequences and alleles for single nucleotide variant (SNV) rs73184087 in NCBI Reference Sequence: NC_000007.14 (Homo sapiens chromosome 7, GRCh38.p14 Primary Assembly). FIG. 1B provides a nucleotide sequence (SEQ ID NO: 5) depicting flanking nucleic acid sequences to the location of rs73184087, in which the major allele A is depicted (bold, underlined).

FIG. 2 shows schematically the relationship between binding of HIF-2α to the G of rs73184087, and the effect of that binding on KMT2E-AS1 expression, stabilization of the KMT2E protein, and PH.

FIGS. 3A-3C. KMT2E-AS1 regulates a gene network driving hypoxic metabolic adaptions and endothelial pathophenotypes. (A) As shown by a heat map representing RNA sequencing of hypoxic human PAECs, knockdown of either KMT2E (top row) and KMT2E-AS1 (middle row) phenocopied each other by reversing the expression of a cohort of Kreb's cycle and metabolism genes that are altered by hypoxia (bottom row). Adjusted p<0.05 for each gene shown. H3K4me3 chromatin immunoprecipitation and sequencing (ChIP-Seq) was also performed in hypoxic vs. normoxic PAECs. By co-analyzing these ChIP-Seq and RNA Seq data, a sub-cohort of these metabolic genes were found to display increases of H3K4me3 marks in hypoxia (fold change in hypoxia is shown for genes with p<0.05). (B) Gene set enrichment analysis (GSEA) of RNA sequencing in (A) revealed enrichment of metabolic and HIF-dependent gene networks. (C) Consistent with (A), ChIP-qPCR (IP: H3K4me3 Ab vs. IgG control) demonstrated H3K4me3 marks at two promoter sites (A:−610 bp, B:−250 bp) of N-MYC downstream-regulated gene 1 (NDRG1). (D-E) Via Seahorse assay, KMT2E-AS1 knockdown reversed the HIF-2α-dependent increase of extracellular acidification rate (ECAR) (D) and reversed the HIF-2α-dependent decrease of baseline oxygen consumption rate (OCR) (E). (F-G) KMT2E-AS1 overexpression increased ECAR (F) but decreased baseline OCR (G). (H-I) KMT2E-AS1 knockdown decreased the hypoxic increase of lactate dehydrogenase (LDH) enzymatic activity (H), a representative measure of glycolysis, while KMT2E-AS1 overexpression increased LDH activity (I). (J) By immunoblot and densitometry, knockdown of KMT2E-AS1 and KMT2E in hypoxic PAECs reversed the increase of VEGF expression in hypoxia, a known HIF-dependent gene. This was consistent with increased H3K4me3 at the VEGFA gene in hypoxia (fold change of 1.48). (K) In human PAECs, as quantified by apoptotic caspase 3/7 activation, KMT2E-AS1 knockdown increased apoptosis in hypoxia (left), while KMT2E-AS1 overexpression inhibited apoptosis under normoxia (right). (L) KMT2E-AS1 knockdown decreased BrdU proliferative potential in hypoxia (left), while forced KMT2E-AS1 expression increased proliferation in normoxia (right). (M-N) By scratch wound healing assay (M), KMT2E-AS1 knockdown decreased PAEC migration under hypoxia (left, N), while overexpression of KMT2E-AS1 promoted migration in normoxia (right, N). (O-P) Knockdown of KMT2E-AS1 in PAECs produced conditioned media that decreased PASMC contraction in gel matrix under hypoxia (O) and as quantified by % contraction (P, left graph). Forced expression of KMT2E-AS1 in PAECs generated conditioned media that increased PASMC contraction under normoxia (right graph). (Q) KMT2E knockdown decreased vasoconstrictive EDN1 under hypoxia (left), while forced KMT2E expression increased EDN1 in normoxia (right). Data represented mean±SEM (*p<0.05, **p<0.01, ****p<0.0001). Scale bars, 200 μm.

FIGS. 4A-4D. G allele of KMT2E SNV rs73184087 binds HIF-2α to control the KMT2E-AS1/KMT2E pair. (A) Among 883 genotyped and imputed SNVs in the PAH discovery cohort within and flanking (+/−200 kb) the IncRNA-KMT2E locus, we identified 59 SNVs with predicted HIF-2α binding to one of either the minor or major SNV alleles. Of those, SNV rs73184087 ranked the highest and met the P-value threshold of 0.000847 (as indicated by the dashed line on the plot). (B) High-throughput chromatin conformation capture (Hi-C) in lung tissue (29) demonstrated long range interactions between SNV rs73184087 and the transcription start site/promoter region of KMT2E-AS1/KMT2E (as indicated by the blue arcs below the graph). A distance-normalized frequency (magenta dots) greater than the threshold of 2.0 by default (green line) defined a significant interaction with a SNV. (C) Via biotin-labeled SNV oligonucleotide incubation with hypoxic PAEC nuclear extracts followed by streptavidin pulldown and immunoblot with densitometry (53), SNV G allele was found to bind HIF-2α more than the A allele. (D) A reporter plasmid was generated by placement of the IncRNA-KMT2E promoter upstream of—and the SNV rs73184087 (A vs. G alleles) downstream of—a secreted luciferase reporter gene. Co-transfection of this plasmid along with an expression plasmid encoding for a constitutively active HIF-2α into HEK293T cells was followed by luciferase quantitation of protein lysates normalized to constitutively secreted alkaline phosphatase (GLuc/SeAP), demonstrating that the G allele increased KMT2E expression more than the A allele. (E) In hypoxic transformed lymphocytes from WSPH Group 1 PAH patients carrying SNV rs73184087 (G/G) vs. (A/A) genotypes, ChIP-qPCR demonstrated enhanced enrichment of SNV after HIF-2α pulldown vs. lymphocytes with A/A alleles. (F) Chromatin conformation capture (3C) assay in three pairs of matched transformed lymphocytes (A/A vs. G/G) from WSPH Group 1 PAH patients. The top diagram shows the design of the 3C assay. PCR primers were designed to detect the fusion ends of given segment pairs in the ligated samples but not non-ligated controls. The fusion-ends were sequenced to confirm ligation products indicative of an interaction between SNV rs73184087 and the IncRNA-KMT2E promoter, irrespective of SNV genotypes. (G-H) 3C assay in human PAECs with SNV A/A genotype defined a ligation product indicative of an interaction between the SNV and promoter (G) but not upstream or downstream of the IncRNA-KMT2E promoter (H). (I-J) After induction of HIF-a by cobalt chloride (50 μM) in transformed lymphocytes from (E-F), G/G genotype more robustly increased both KMT2E-AS1 (I) and KMT2E (J) vs. the A/A genotype. Data represented mean±SEM (C,E,I-J) (*p<0.05, **p<0.01).

FIGS. 5A and 5B. E22 knockout mice display decreased KMT2E and H3K4me3 along with disease improvement in mouse models of WSPH Groups 1 an 3 PH. (A) Via CRISPR/Cas9 genome editing, mice were generated that were genetically deficient in a conserved 500 bp sequence (denoted A-D) shared between human KMT2E-AS1 and mouse E22. (B) By FISH and IF staining, hypoxic E22 knockout (KO) mice with AD deletion showed decreased E22 and KMT2E expression in CD31+ lung endothelial cells. (C-F) Via in situ fluorescent microscopy, similar to normoxic wildtype (WT) mice, hypoxic E22 (KO) mice showed reduced H3K4me3 (red; C,E) expression in CD31+ PAECs (green) as compared to hypoxic wildtype (WT) controls. Both E22 KO and normoxic WT controls also displayed reduced expression of the proliferation marker Ki67 (red, D) in the endothelium (F). (G-H) By a-SMA stain (white, C-D), vascular remodeling was reduced in hypoxic E22 KO mice as compared to hypoxic WT, with decreased pulmonary vascular thickness (G) and muscularization (H). (I-J) Hypoxic E22 KO mice showed reduced right ventricular systolic pressure (I) via right heart catheterization and reduced RV remodeling (RV/body weight ratio, J). (K) Using FISH and fluorescence microscopy, E22 and KMT2E were down-regulated in lung CD31+ endothelium of interleukin-6 transgenic (IL-6 Tg) mice crossed onto E22 KO (AD deletion) mice. (LO) Hypoxic IL-6 Tg; E22 KO mice displayed reduced H3K4me3 in mouse lung vasculature as compared to hypoxic IL-6 Tg PAH mice (L,N). These E22 KO mice also displayed decreased endothelial Ki67 expression, indicative of downregulated proliferation (M,O). (P-Q) The E22 KO mice (white, L-M) displayed reduced vessel remodeling with decreased vessel thickness (P) and muscularization (Q). (R-S) PH was alleviated in E22 KO mice, showing improved RVSP (R) and RV remodeling (S). Data showed mean±SEM (*p<0.05, **p<0.01, ***p<0.001, ****p<0.0001). Scale bars, 50 μm.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values.

As used herein, “a” and “an” refer to one or more.

The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” those stated elements or steps. For definitions provided herein, those definitions refer to word forms, cognates and grammatical variants of those words or phrases.

As used herein, the terms “patient” or “subject” refer to members of the animal kingdom including but not limited to human beings and “mammal” refers to all mammals, including, but not limited to human beings.

“Treatment” in the context of a disease or disorder, a marker for a disease or a disorder, or a symptom of a disease or disorder, can refer to a clinically-relevant and/or a statistically significant decrease or increase in an ascertained value for a clinically-relevant marker from outside a normal range towards, or to, a normal range. The decrease or increase can be, for example, at least 10%, at least 20%, at least 30%, at least 40%, or more, to a level accepted as either a therapeutic goal, or a level within the range of normal for an individual without such disease or disorder, or, in the case of a lowering of a value, to below the level of detection of an assay. The decrease or increase can be to a level accepted as within the range of normal for an individual without such disease or disorder, which can also be referred to as a normalization of a level. The reduction or increase can be the normalization of the level of a sign or symptom of a disease or disorder, that is, a reduction in the difference between the subject level of a sign of the disease or disorder and the normal level of the sign for the disease or disorder (e.g., to the upper level of normal when the value for the subject must be decreased to reach a normal value, and to the lower level of normal when the value for the subject must be increased to reach a normal level).

As used herein, the terms “cell” and “cells” refer to any types of cells from any animal, such as, without limitation, rat, mouse, monkey, and human. For example and without limitation, cells can be progenitor cells, e.g., pluripotent cells, including stem cells, induced pluripotent stem cells, multipotent cells, or differentiated cells, such as endothelial cells and smooth muscle cells. “Cells” may be in vivo, e.g., as part of a tissue or organ, or in vitro, such as a population of cells, such as, for example, a population of cells enriched for a specific cell type, such as, without limitation, a progenitor cell or a stem cell.

“Therapeutically effective amount,” as used herein, can include the amount of a gene editing reagent, as described herein that, when administered to a subject having a disease, can be sufficient to effect treatment of the disease (e.g., by diminishing, ameliorating or maintaining the existing disease or one or more symptoms of disease). The “therapeutically effective amount” may vary depending on the gene editing reagent, how the composition is administered, the disease and its severity, and the history, age, weight, family history, genetic makeup, the types of preceding or concomitant treatments, if any, and other individual characteristics of the subject to be treated.

A “therapeutically-effective amount” can also include an amount of an agent that produces a local or systemic effect at a reasonable benefit/risk ratio applicable to any treatment. A gene editing reagent, employed in the methods described herein may be administered in a sufficient amount to produce a reasonable benefit/risk ratio applicable to such treatment.

The phrase “pharmaceutically-acceptable carrier” as used herein can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier can be “acceptable” in the sense of being compatible with the other ingredients of the formulation and not injurious to the subject being treated. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (24) other non-toxic compatible substances employed in pharmaceutical formulations.

By “expression” or “gene expression,” it is meant the overall flow of information from a gene (without limitation, a functional genetic unit for producing a gene product, such as RNA or a protein in a cell, or other expression system encoded on a nucleic acid and comprising: a transcriptional promoter and other cis-acting elements, such as response elements and/or enhancers; an expressed sequence that typically encodes a protein (open-reading frame or ORF) or functional/structural RNA, and a polyadenylation sequence), to produce a gene product (typically a protein, optionally post-translationally modified or a functional/structural RNA). By “expression of genes under transcriptional control of,” or alternately “subject to control by,” a designated sequence, it is meant gene expression from a gene containing the designated sequence operably linked (functionally attached, typically in cis) to the gene. The designated sequence may be all or part of the transcriptional elements (without limitation, promoters, enhancers and response elements), and may wholly or partially regulate and/or affect transcription of a gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when an amount of the respective inducer is administered to the expression system (e.g., cell) effective to cause expression of the gene.

As used herein, the term “nucleic acid” refers to deoxyribonucleic acids (DNA) and ribonucleic acids (RNA). Nucleic acid analogs include, for example and without limitation: 2′-O-methyl-substituted RNA, locked nucleic acids, unlocked nucleic acids, triazole-linked DNA, peptide nucleic acids, morpholino oligomers, dideoxynucleotide oligomers, glycol nucleic acids, threose nucleic acids and combinations thereof including, optionally ribonucleotide or deoxyribonucleotide residue(s). Herein, “nucleic acid” and “oligonucleotide” which is a short, single-stranded structure made of up nucleotides, in reference to nucleic acids and nucleic acid analogs, are used interchangeably. An oligonucleotide may be referred to by the length (i.e. number of nucleotides) of the strand, through the nomenclature “-mer”. For example, an oligonucleotide of 22 nucleotides would be referred to as a 22-mer.

A “nucleic acid analog” is a composition comprising a sequence of nucleobases arranged on a substrate, such as a polymeric backbone, and can bind DNA and/or RNA by hybridization by Watson-Crick, or Watson-Crick-like hydrogen bond base pairing. Non-limiting examples of common nucleic acid analogs include peptide nucleic acids, such as yPNA, morpholino nucleic acids, phosphorothioates, locked nucleic acid (2′-O-4′-C-methylene bridge, including oxy, thio or amino versions thereof), unlocked nucleic acid (the C2′-C3′ bond is cleaved), 2′-O-methyl-substituted RNA, threose nucleic acid, glycol nucleic acid, etc.

Hypoxia signaling via HIF is implicated in a number of conditions (Lee J W, et al. Hypoxia signaling in human diseases and therapeutic targets. Exp Mol Med. 2019 June 20;51(6):1-13). H3K4 methylation is an evolutionarily conserved histone modification that marks active transcription and is highly enriched at the promoter region and transcription start site (see, e.g., Hyun K, et al. Writing, erasing and reading histone lysine methylations. Exp Mol Med. 2017 April 28;49(4):e324). As shown herein, activation of KMT2E, results in a significant endothelial metabolic shift, and selective binding of HIF-2α to the site of the G-allele at rs73184087 results in increased expression of KMT2E-AS1, and consequently stabilization of the KMT2E protein and follow-on H3K4 trimethylation with its consequential metabolic shifts in endothelial cells. Certain diseases, such as PH/PAH, would benefit from down-regulation of the KMT2E-AS1, and others may benefit from up-regulation of expression of KMT2E-AS1. As discussed above, PH and PAH are directly influenced by HIF signaling, and can be ameliorated in patients having one or more Gs at rs73184087 by converting the one or more Gs to a different base. Likewise, HIF-2α inhibitors have been approved for Von Hippel Lindau disease for tumor regression, and in patients having one or more Gs at rs73184087, the disease may be treated by converting the one or more Gs to a different base. Certain other conditions may be treated by the converse, by up-regulating expression of KMT2E-AS1 and therefore H3K4 trimethylation, by conversion of an A, C, or T at rs73184087 to a G, such as, without limitation: anemia, such as anemia in chronic kidney disease (see, e.g., Wyatt C M, Drüeke T B. HIF stabilization by prolyl hydroxylase inhibitors for the treatment of anemia in chronic kidney disease. Kidney Int. 2016 November;90(5):923-925); peripheral vascular disease and limb ischemia, to increase blood supply and angiogenesis; and ischemic preconditioning and remote ischemic preconditioning, e.g., repeated cessation and restoration of blood flow in a particular limb, thus inducing hypoxia and HIF induction, to protect against hypoxic or ischemic injury during surgery. As discussed herein, the treatment of modifying the nucleobase at rs73184087 may be administered systemically, or locally to an affected organ, tissue, limb, system, etc.

Methods of treating a disease in a patient having one or more Gs at rs73184087 is provided in which expression of HIF-2α is elevated above normal. Alternatively a method of reducing H3K4 methylation in a patient having one or more Gs at rs73184087 is provided. The methods comprise deleting the one or more of the Gs at rs73184087 or substituting the one or more Gs at rs73184087 with A, T, or C in a cell, tissue, or organ of the patient. Such diseases include, for example and without limitation: pulmonary hypertension, pulmonary arterial hypertension, myocardial infarct, ischemia/reperfusion injury, ischemia, valvular heart disease, congestive heart failure, stroke, cancer, thrombus, embolism, and disease-related ischemia, such as with sepsis.

Methods of treating a patient having one or more As, Ts, or Cs at rs73184087 is provided in which expression of HIF-2α is below normal. Alternatively a method of increasing H3K4 methylation in a patient having one or more As, Ts, or Cs at rs73184087 is provided. Diseases or conditions amenable to treatment that increases H3K4 methylation include, for example and without limitation: an anemia, such as anemia in chronic kidney disease; peripheral vascular disease and limb ischemia, to increase blood supply and angiogenesis; and ischemic preconditioning and remote ischemic preconditioning. The methods comprise substituting the one or more As, Ts, or Cs at rs73184087 with G in a cell, tissue, or organ of the patient.

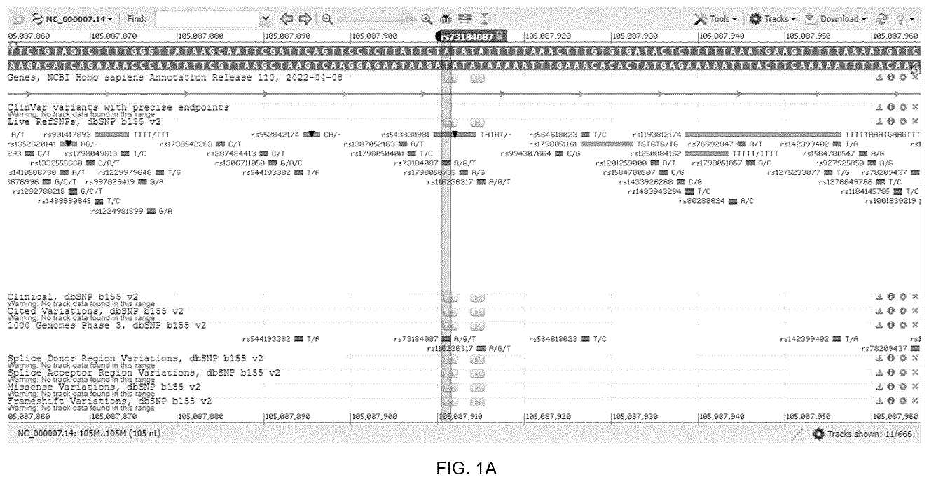

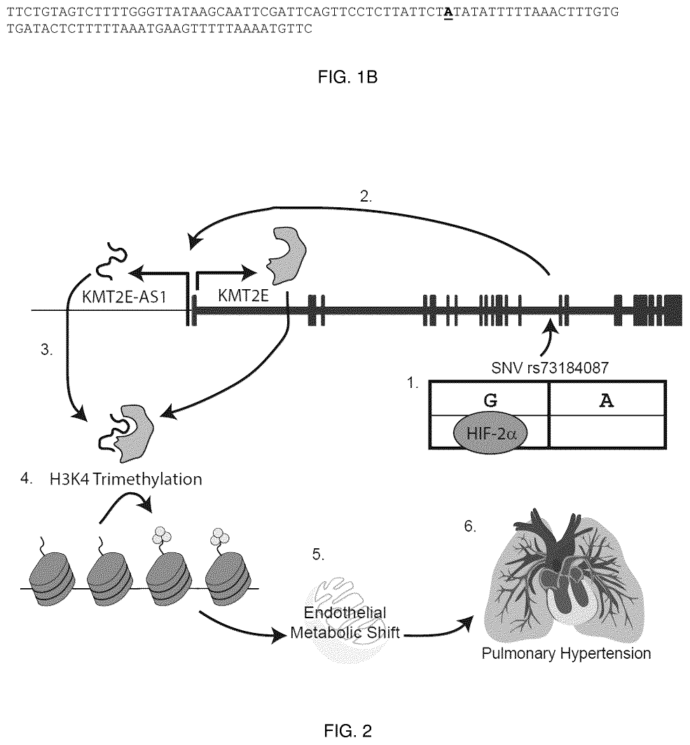

The polymorphism rs73184087 and its flanking sequences are depicted in FIG. 1A (dbSNP) and FIG. 1B. Suitable guide RNA (gRNA) sequences and PAM sequences for gene editing, for example and without limitation, CRISPR/CAS9-based editing, base editing, or prime editing of the minor, risk, allele G to a different nucleobase, such as to the major allele A, at rs73184087 can be determined and optimized based on this sequence.

Gene editing in any form may be used to modify the G at rs73184087. A G at rs73184087 may be deleted (it falls within an intron), or more preferably changed to the major allele A, or C or T. A CRISPR-CAS9 editing (Clustered Regularly Interspaced Short Palindromic Repeats (CRISPR)-CRISPR-associated protein 9 (Cas9)) system, single base editing, or prime editing, as examples of gene editing methods, may be used to remove or edit a G nucleobase at rs73184087 to a different nucleobase. CRISPR-Cas9 may be used to inactivate or correct a gene, or base editors or prime editors, including cytosine base editors (CBEs, for converting C→T) and adenine base editors (ABE, for converting A→G). The CRISPR-Cas9 system as well as single base editors include a guide RNA (gRNA) or single guide RNA (sgRNA) and a CRISPR-associated protein 9 (Cas9) nuclease. Identification of the DNA target strand, and methods of implementing a change in the target DNA (e.g., gene knock out in the target DNA strand, knock-in of a desired sequence, or base substitutions) are within the abilities of one of ordinary skill in the art.

The non-target DNA strand includes a specific protospacer adjacent motif (PAM) in order for the gRNA to bind to the target DNA strand. The PAM is a short nucleotide motif that is found 3′ to the target site. For the CRISPR-Cas9 system, the PAM may be 5′-NGG-3′, where N is any nucleotide and G is guanine. The Cas9 nuclease cuts 3 to 4 nucleotides upstream of the PAM sequence. The locations in the genome that can be targeted by different Cas proteins are limited by the locations of the PAM sequences and are known to those of ordinary skill in the art.

In Crispr-Cas9 editing, when the Cas9 nuclease binds with the PAM and the gRNA binds with the target DNA strand, a double-strand break is caused in the gRNA sequence. Endogenous repair mechanisms, such as non-homologous end joining (NHEJ), microhomology-mediated end joining (MMEJ), or homologous directed repair (HDR), are triggered by the double-strand break and result in a gene knock out in the target DNA strand or a knock-in of a desired sequence if a DNA template is present. The DNA template includes the desired sequence, which is flanked by sequences that are homologous to the region upstream and downstream of the double-stranded break.

The gRNA includes a Crispr RNA (crRNA), which is a 17-20 nucleotide sequence that is complementary to the target DNA strand, and a tracrRNA, which serves as a binding scaffold for the Cas 9 nuclease. The crRNA and the tracrRNA may exist as two separate RNA molecules. Alternatively, the sgRNA may comprise both the crRNA sequence and the tracrRNA sequence, where the crRNA sequence is fused to the scaffold tracrRNA sequence. gRNAs of base editing methods as described below, have canonical structures specific to each technique. One of ordinary skill in the art would select a gRNA or sgRNA that maximizes the on-target DNA cleavage efficiency, while also minimizing unintentional off-target binding and cleavage effects (see, Konstantakos et al. “CRISPR-Cas9 gRNA efficiency prediction: an overview of predictive tools and the role of deep learning. Nucleic Acids Res., 2022, 50(7):3616-3637 and “The Complete Guide to Understanding CRISPR sgRNA”, Synthego, 2023, www.synthego.com/guide/how-to-use-crispr/sgrna).

Alternatively, a base editing system may be used to convert a G to another nucleobase. Base editing is a genome-editing technique that uses DNA base editors to directly generate precise point mutations without generating a double-strand break without double-strand breaks. The DNA base editors may comprise fusions between a catalytically dead Cas9 (dCas9) or a nickase Cas9 (nCas9) fused to a single-stranded DNA (ssDNA)-specific deaminase and a single guide RNA (sgRNA). The d/nCas9 recognizes a specific sequence named protospacer adjacent motif (PAM) and the DNA unwinds thanks to the complementarity between the sgRNA and the DNA sequence usually located upstream of the PAM (“protospacer”). Then, the opposite DNA strand is accessible to the deaminase that converts the bases located in a specific DNA stretch of the protospacer (see, e.g., Antoniou P, Miccio A, Brusson M. Base and Prime Editing Technologies for Blood Disorders. Front Genome Ed. 2021 Jan. 28;3:618406). Upon binding of the DNA base editor to the target DNA strand, base pairing between the sgRNA and the target DNA strand results in the displacement of a small segment of ssDNA as an “R-loop”. The DNA bases within the ssDNA are therefore substrates for deamination and are subsequently modified by the deaminase enzyme. The DNA base editor may be a cytosine base editor (CBE), which converts a C/G base pair into a T/A base pair or an adenine base editor (ABE) which converts an A/T base pair into a G/C base pair (see, e.g., Qi et al. “Base Editing Mediated Generation of Point Mutations Into Human Pluripotent Stem Cells for Modeling Disease”, Frontiers in Cell and Developmental Biology, 2020, 8(590581):1-12; Nishida K, et al. Targeted nucleotide editing using hybrid prokaryotic and vertebrate adaptive immune systems. Science. 2016 Sep. 16;353(6305):aaf8729; Komor AC, et al. Programmable editing of a target base in genomic DNA without double-stranded DNA cleavage. Nature. 2016 May 19;533(7603):420-4; and Gaudelli N M, et al. Programmable base editing of A·T to G·C in genomic DNA without DNA cleavage. Nature. 2017 Nov. 23;551 (7681):464-471).

Another method of editing SNV is prime editing, which is disclosed in U.S. Pat. No. 11,447,770 B1, incorporated herein by reference for its technical disclosure, and related publications (see also, International Patent Publication No. WO2020191242 A1 and Anzalone AV, et al. Search-and-replace genome editing without double-strand breaks or donor DNA. Nature. 2019 December;576(7785):149-157). Prime editors (PEs), including a complete description of pegRNA are provided in those references, as well as methods of in vivo delivery of prime editor materials, such as viral vectors, e.g., AAV particles encoding prime editors, are described in that patent publication and related publications.

Prime editing is a “search and replace” gene editing method in which Moloney Murine Leukemia Virus Reverse Transcriptase (M-MLV RT) is fused to the C-terminus of Cas9 H840A nickase, The fusion enzyme is installs targeted insertions, deletions, and all possible base-to-base conversions using a prime editing guide RNA (pegRNA). The pegRNA directs the nickase to the target site by homology to a genomic DNA locus. The longer pegRNA also encodes a primer binding site (PBS) and the desired edits on an RT template. Prime editing has gone through a number of versions. In PE1, the pegRNA directs the Cas9 nickase to the target sequence where it nicks the non-target strand and generates a 3′ flap. The 3′ flap binds to the primer binding site (PBS) of the pegRNA and the desired edit is incorporated into the DNA by reverse transcription. The edited DNA strand displaces the unedited 5′ flap and the resulting heteroduplex is resolved by the cell's mismatch repair (MMR) system. Alternatively, the edited 3′ flap may be excised and the target sequence will remain unchanged but available as a substrate for another round of prime editing.

In the PE2 system, mutations were introduced into the RT enzyme to increase activity, enhance binding between the template and PBS, increase processivity, and improve thermostability. PE3 uses the PE2 Cas9 nickase-pentamutant RT fusion enzyme and pegRNA plus an additional simple sgRNA, which directs the Cas9 nickase to nick the unedited strand at a nearby site. The newly edited strand is then favored as the template for repair during heteroduplex resolution. The process of double nicking, however, increases indel formation slightly. Designing the sgRNA with a spacer that only binds the edited strand, as in the PE3b system, guides nicking of the unedited strand only after the edit has occurred. PE4 and PE5 also have been described (Chen P J, et al. Enhanced prime editing systems by manipulating cellular determinants of editing outcomes. Cell. 2021 Oct. 28;184(22):5635-5652.e29). Plasmids useful for performing prime editing are commercially-available from Addgene (www.addgene.org/crispr/prime-edit/). “Prime editing” includes all variations of prime editing methods, including, without limitation, PE1, PE2, PE3, PE3b, PE4, and PE5 versions. pegRNA includes variations thereof for use in the many variations of prime editing, such as, without limitation, epegRNA (Nelson J W, et al. Engineered pegRNAs improve prime editing efficiency. Nat Biotechnol. 2022 March;40(3):402-410).

Computer-based tools have been developed for automated generation of pegRNA (see, e.g., Morris et al. Automated design of CRISPR prime editors for 56,000 human pathogenic variants. iScience. 2021 Oct. 30;240 1):103380, and the tool, Prime Editing Design Tool, for identification of useful pegRNAs is provided at primeedit.nygenome.org/; Hwang G H, et al. PE-Designer and PE-Analyzer: web-based design and analysis tools for CRISPR prime editing. Nucleic Acids Res. 2021 Jul. 2;49(W1):W499-W504; Hsu J Y, et al. PrimeDesign software for rapid and simplified design of prime editing guide RNAs. Nat Commun. 2021 Feb. 15;12(1)1034; and Chow R D, et al. A web tool for the design of prime-editing guide RNAs. Nat Biomed Eng. 2021 February;5(2):190-194). pegRNAs may contain a protospacer sequence for recognizing the target sequence, a reverse transcriptase template (RTT) that contains the desired edit, and a primer binding site (PBS) for the activation of reverse transcriptase. As above, several types of PEs have been developed and different gRNAs can be used, depending on the type of prime editor (e.g. PE2 vs PE3). For example, in contrast with PE2, which requires only a pegRNA, PE3 also requires a nicking guide RNA (ngRNA) to increase the prime editing efficiency (Hwang G H, et al, Nucleic Acids Res. 2021 Jul. 2;49(W1):W499-W504). Although pegRNAs and ngRNAs, when applied, can be developed by a person of ordinary skill without use of a computer, that person may employ computational tools, such as those described above, to effectively design a pegRNA, and other useful reagents, for prime editing.

The CRISPR-Cas 9, base editing, and prime editing, necessary components, e.g., gRNA, template, pegRNA, ngRNA, nucleic acid encoding Cas9, Cas9 nickase, or Cas9 fusion proteins, etc. may be delivered by any effective means, e.g. by a viral delivery vehicle or a non-viral delivery vehicle, or the delivery may be a physical cell manipulation technique. The transferred material may take any useful form, but may include a DNA plasmid or recombinant viral genome containing sequences for expression of necessary reagents; and mRNA for translation of the reagents, along with suitable guide RNA and other useful nucleic acid reagents, such as ngRNA.

The viral delivery method may be achieved, for example and without limitation, through the use of recombinant adeno-associated virus (AAV) vectors, adenoviral (Ad) vectors, or lentiviral vectors as are broadly-known (see, e.g., Zhi S, Dual-AAV delivering split prime editor system for in vivo genome editing. Mol Ther. 2022 Jan. 5;30(1):283-294). For example, 293 cells (e.g., HEK293T or HEK293 cells) may be used to create viral particles that contain nucleic acid for expression of the components for, e.g., CRISPR/Cas9 editing, base editing, or prime editing, which then infect the target cells.

Non-viral delivery methods of the components may include, but are not limited to liposomes, polymeric nanoparticles, lipid nanoparticles, gold nanoparticles, inorganic nanoparticles, lipoplexes, polyplexes, cell-penetrating peptides, and combinations thereof (see, Synthego “Delivery of CRISPR-Cas9: Cargo, Vehicles, Challenges, and More”, 2023, www.synthego.com/blog/delivery-crispr-cas9). For example, DNA plasmids expressing both Cas9 and gRNA may be delivered through polymeric nanoparticles, as shown in Zhang et al. (“Robust genome editing in adult vascular endothelium by nanoparticle delivery of CRIPSR-Cas9 plasmid DNA”, 2022, Cell Reports. 38(110196):1-21). RNA alternatively may be delivered through the use of lipid nanoparticles (see e.g., International Patent Application Publication No. WO 2019/204451 A1; International Patent Application Publication No. WO 2022/236093 A1 or WO 2022260772 A1). Physical cell manipulation techniques to deliver the components may include, but are not limited to electroporation, microfluidics, microinjection, hydrodynamic delivery, and combinations thereof.

Delivery of the gene editing components, such as genes encoding required proteins, and genes for expressing gRNAs or pegRNAs to vascular tissue or diseased or injured tissue may be accomplished through various delivery routes using suitable delivery vehicles. For delivery to the lungs and airway, a formulation may be sprayed, aerosolized, or delivered as a fine powder. Delivery to vasculature may be achieved through direct injection or through use of a delivery catheter device, such as a balloon catheter.

Induced pluripotent stem cells (iPSC) are artificial stem cells that are generated from adult, terminally differentiated somatic cells. iPSC technology allows cells from any donor (e.g., skin cells or blood cells) to be reprogrammed into an embryonic-like pluripotent state. Like embryonic stem cells (ESCs), iPSCs can typically proliferate and self-renew indefinitely in vitro and differentiate into derivatives of all three primary germ layers (e.g., ectoderm, mesoderm, and endoderm) as well as germ cells that give rise to the gametes. iPSCs are generated from somatic cells through the ectopic co-expression of defined pluripotency factors. Methods of generation of iPSCs are broadly known to those of ordinary skill (see, e.g., Bragança J, Lopes J A, Mendes-Silva L, Almeida Santos J M. Induced pluripotent stern cells, a giant leap for mankind therapeutic applications. World J Stem Cells. 2019 Jul. 26;11(7):421-430 and Liu G, David B T, Trawczynski M, Fessler R G. Advances in Pluripotent Stem Cells: History, Mechanisms, Technologies, and Applications. Stem Cell Rev Rep. 2020 February;16(1):3-32). According to one aspect or embodiment of the present invention, iPSCs are provided. The iPSCs may be human and may be derived from human cells heterozygous or homozygous for the minor allele G at rs73184087 (comprising one or two G alleles for rs73184087). Among other potential uses, iPSCs having such generic markers may be utilized in a drug-development capacity to screen for APIs for treatment of IncRNA KMT2E-AS1 activation/stabilization of KMT2E, and therefore conditions such as PH, e.g. PAH. iPSCs can be differentiated into various cell types, including terminal cell types by suitable culture methods as are broadly-known in the art. iPSCs having the and cells differentiated therefrom can serve as a source of cells for use inn screening the efficacy of APIs for treatment of PH or other hypoxia-related diseases. The cells, homozygous for G at rs73184087, optionally differentiated into a specific cell type, such as endothelial cells (see, e.g., Jang S, Collin de I'Hortet A, Soto-Gutierrez A. Induced Pluripotent Stem Cell-Derived Endothelial Cells: Overview, Current Advances, Applications, and Future Directions. Am J Pathol. 2019 March;189(3):502-512), may be used in a suitable cell culture vessel, such as a multi-well plate (e.g. 96-well), to screen candidate APIs for efficacy in treating a hypoxia-related disease, such as PH or PAH. For example, cells can be cultured under hypoxic conditions, e.g., less O₂ than is found in air (normoxic) such as at 5% O₂, and an API's ability to prevent downstream events resulting from KMT2E stabilization by KMT2E-AS1, such as lysine trimethylation on histone 3 by increased activity of H3K4me3, e.g. detected by immunoblot.

### Example 1

Long non-coding RNAs (IncRNAs) can exert regulatory activity across genomic, epigenetic, metabolic domains. IncRNAs are single stranded RNAs that affect cellular function by complexing with chromosomal DNA, RNAs, or proteins, and/or may prevent miRNA binding to target mRNAs. IncRNAs are dysregulated in PH, and certain IncRNAs are controlled by PH triggers, such as hypoxia. Characterization of IncRNAs in pulmonary vascular cells has been limited, and functional data regarding their roles in PH is just emerging. Yet, the majority of IncRNAs do not carry full sequence conservation in mammals, making it challenging to translate in vivo IncRNA biology between rodents and humans.

Combining insights of genetic epidemiology with molecular mechanism, we identified an IncRNA-protein pair, governed in part by an endogenous human single nucleotide variant (SNV), that carries crucial epigenetic and metabolic functions in endothelial cells and controls PH manifestation in vivo.

In further detail, hypoxic reprogramming of vasculature relies upon genomic, epigenetic, and metabolic circuitry, but the control points are unknown. In pulmonary arterial hypertension (PAH), a disease driven by HIF-dependent vascular dysfunction, HIF-2α promoted expression of neighboring genes, IncRNA KMT2E-AS1 and histone lysine N-methyltransferase 2E (KMT2E). KMT2E-AS1 stabilized KMT2E protein to alter epigenetic histone 3 lysine 4 trimethylation (H3K4me3), driving metabolic and pathogenic endothelial activity. We identified a significant association between rs73184087, a single nucleotide variant (SNV) within a KMT2E intron, and disease risk in PAH discovery (N=694 vs. 1,560 controls) and replication (N=96 vs. 401 controls) patient cohorts and in a global meta-analysis (N=2,181 vs. N=10,060 controls). Mechanistically underlying this association, this SNV displayed allele (G)-specific association with HIF-2α, engaging in long-range chromatin interactions and inducing the IncRNA-KMT2E tandem in hypoxic (G/G) cells (see, FIG. 2). In vivo, KMT2E-AS1 deficiency protected against PAH, as did pharmacologic inhibition of histone methylation. Thus, the KMT2EAS1/KMT2E pair orchestrates across convergent multiome landscapes and represents key clinical targets in vascular pathobiology. Background and supportive information and data is provided in priority U.S. Provisional Patent Application No. 63/334,377 filed Apr. 25, 2022, which is hereby incorporated by reference in its entirety.

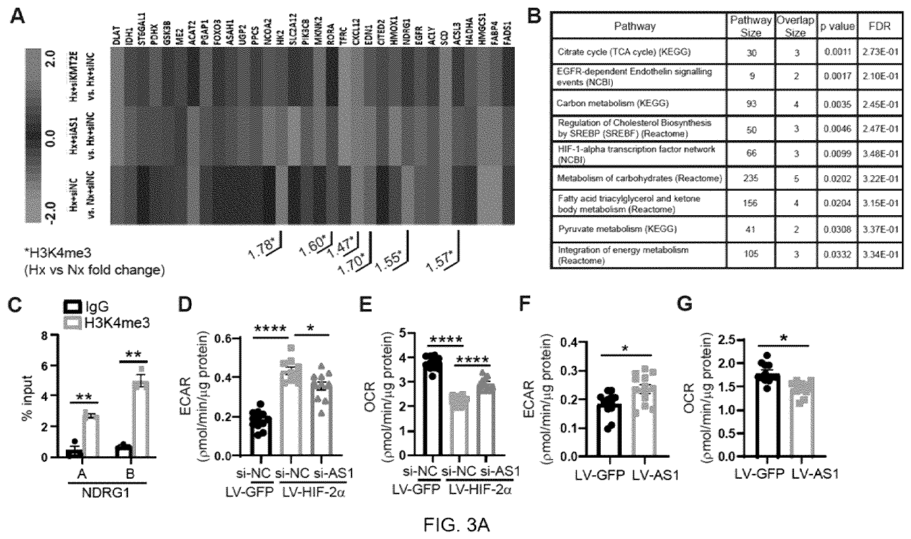

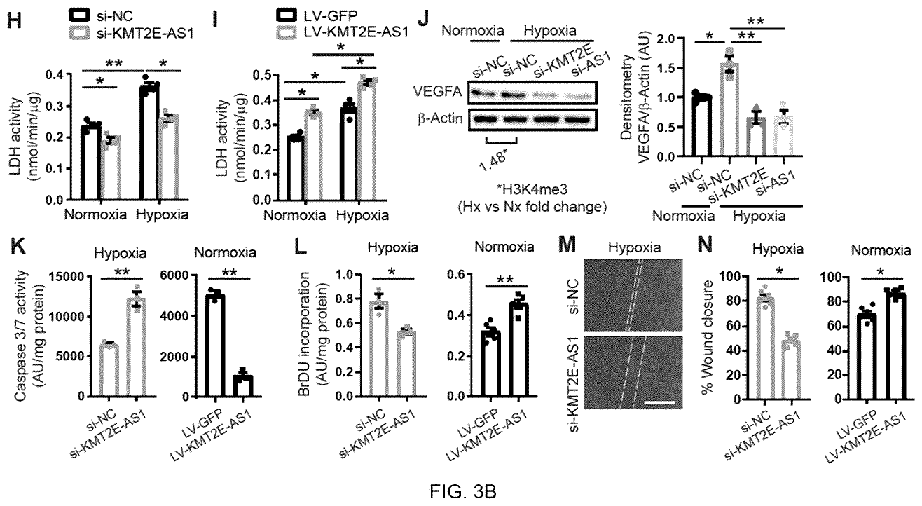

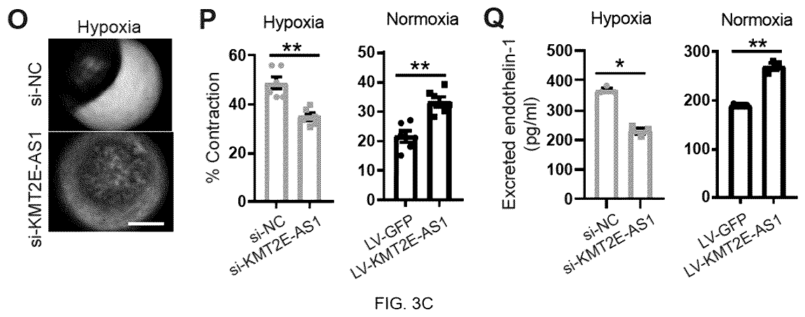

KMT2E-AS1 drives hypoxic metabolic reprogramming. As KMT2E-AS1 acts in conjunction with KMT2E to regulate epigenetic H3K4me3, we sought to define the transcriptional alterations under this IncRNA's control during hypoxic endothelial reprogramming. In PAECs, siRNA knockdown of either KMT2E or KMT2E-AS1 phenocopied each other by reversing the expression of a cohort of Kreb's cycle and metabolism genes that were altered by hypoxia (FIG. 3A (A)). Gene set enrichment analysis (GSEA) of these RNA sequencing results revealed enrichment of metabolic and HIF-dependent gene networks (FIG. 3A (B)). To determine which of these changes are controlled directly by H3K4me3, H3K4me3 chromatin immunoprecipitation and sequencing (ChIP-Seq) was performed in hypoxic vs. normoxic PAECs. By co-analyzing ChIP-Seq and RNA Seq data, we defined a subcohort of these HlFdependent and metabolism-specific transcripts under the control of H3K4me3 (FIG. 3A (A)), with independent confirmation of NDRG1 by ChIP-qPCR as one such KMT2E-AS1- and H3K4me3- dependent gene (FIG. 3A (C)).

Consistent with this IncRNA's control over HIF-dependent metabolism, KMT2E-AS1 knockdown mitigated the hypoxic induction of extracellular acidification rate (ECAR), a representative measure of glycolysis, as well as concomitant lactate dehydrogenase (LDH) enzymatic activity (FIGS. 3A and 3B (D, H)); forced KMT2E-AS1 expression increased ECAR and LDH activity (FIGS. 3A and 3B (F, I)). Moreover, in cultured PAECs, KMT2E-AS1 knockdown increased baseline oxygen consumption rate (OCR) (FIG. 3A (E)), while forced KMT2E-AS1 decreased OCR indices (FIG. 3A (G)). Furthermore, representing a canonical HIF-2α-dependent and angiogenic factor acting in concert with these metabolic changes, vascular endothelial growth factor (VEGFA) was up-regulated by hypoxia, consistent with an increase of H3K4me3 at its gene locus (FIG. 3B (J)). Importantly, both KMT2E-AS1 and KMT2E knockdown decreased VEGFA expression. Taken together, these data demonstrate that KMT2E-AS1 regulates a gene network that decreases oxidative metabolism, increases glycolysis, and controls hypoxic PAEC adaptation.

KMT2E-AS1 promotes endothelial pathophenotypes of PH. Stemming from these metabolic reprogramming events, KMT2E-AS1 drove endothelial pathophenotypes causatively linked to HIF biology and PH. Knockdown of KMT2E-AS1 increased PAEC apoptotic potential in hypoxia, while forced expression of KMT2E-AS1 via lentiviral transduction decreased apoptosis in normoxia (FIG. 3B (K)). Parallel quantification of BrdU incorporation, KMT2E-AS1 knockdown decreased PAEC proliferation in hypoxia, while forced expression increased proliferation in normoxia (FIG. 3B (L)). Thus, KMT2E-AS1 is necessary and sufficient to promote PAEC proliferation. Consistent with these alterations in cell survival and proliferative capacity, by wound closure assay in vitro, KMT2E-AS1 knockdown decreased PAEC migration in hypoxia, while forced expression increased such activity (FIG. 3B (M, N)). Similarly, by quantifying gel contraction as a surrogate of smooth muscle cell contraction when exposed to PAEC-conditioned media, we found that KMT2E-AS1 knockdown in hypoxic PAECs generated conditioned media that decreased the level of contraction seen in hypoxia, while forced expression of KMT2E-AS1 in PAECs increased contraction in normoxia (FIG. 3C (O, P)). Consistent with these alterations in vasomotor activity, KMT2E-AS1 knockdown decreased secreted endothelin-1 (EDN1) in hypoxia, while forced expression of this IncRNA increased endothelin-1 in normoxia (FIG. 3C (Q)). As a result, we found that KMT2E-AS1 inhibits PAEC apoptosis as well as enhances proliferation, migration, and vasomotor tone, consistent with a role that promotes endothelial dysregulation and PAH.

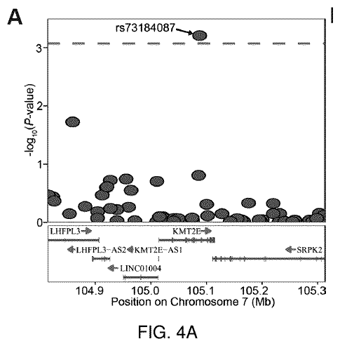

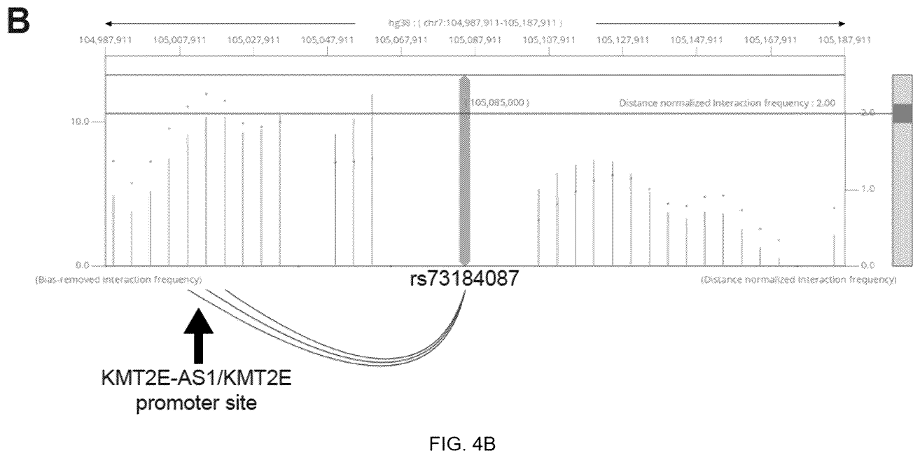

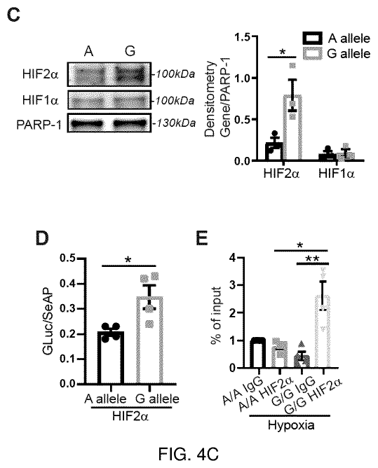

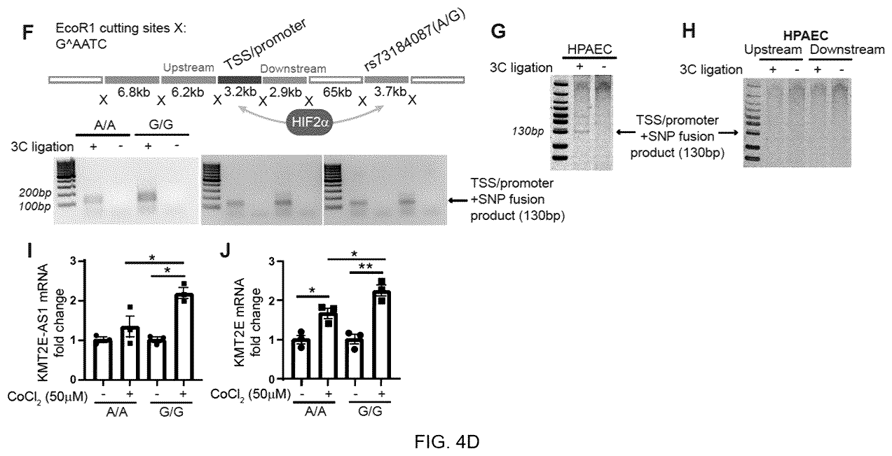

Enrichment of KMT2E SNV rs73184087 (G) allele in WSPH Group 1 PAH. Genetic control of HIF-2α activity can be facilitated by single nucleotide variants (SNVs) that alter transcription factor binding sites. SNVs have been identified to alter HIF binding sites even outside canonical promoter regions with consequent disruptions of long-range genomic interactions with active promoter sites. Thus, we screened for such SNVs relevant to this IncRNAKMT2E locus within a previously reported WSPH Group 1 PAH discovery cohort (“PAH Biobank”) of European-descent (N=694) subjects vs. non-diseased controls (N=1,560). Among the 883 genotyped and imputed SNVs (with minor allele frequency>1%) within and flanking (+/−200 kb) the KMT2E-AS1-KMT2E tandem locus, we prioritized 59 SNVs with predicted HIF-2α binding to one of either the minor or major alleles using position weight matrices (PWMs) derived from HIF-2α chromatin immunoprecipitation sequencing. Among these SNVs, we observed a novel, significant association for rs73184087 with the risk for developing PAH, with the G allele conferring an adjusted odds ratio (OR) of 1.87 (95% CI:1.31-2.67; p=6.2×10⁻⁴) in the discovery cohort (FIG. 4A (A)). Correspondingly, PWM scoring predicted more robust HIF-2α binding to the risk allele (G) of this SNV (Log-odds score 10.01, P<10-8) vs. the ancestral allele (A) (Log-odds score 4.56). We then replicated the SNV association with disease risk in a second, independent PAH cohort of European-descent subjects from the University of Pittsburgh Medical Center (UPMC cohort, Table S6, N=96 cases vs. N=401 non-PAH controls (adjusted OR 2.51 [95% CI:1.26-5.02]; p=0.009). Finally, we replicated the association of this SNV in a global meta-analysis of five cohorts (N=2,181 cases vs. N=10,060 controls; total N=12,241) including the PAH Biobank, UPMC cohort, and three additional European cohorts from a prior study (Rhodes et al., 2019b) (OR=1.48 [95% CI: 1.03-2.11], p=0.03). Based on the robust association with disease risk, this SNV was further characterized by functional validation.

SNV rs73184087 displays allele-specific binding to HIF-2α and long-range interaction with the shared IncRNA-KMT2E promoter. SNV rs73184087 is located at a KMT2E intronic site 75 kB downstream of the IncRNA-KMT2E shared promoter. Based on prior-capture Hi-C mapping in lung tissue, we found a significant long-range genomic interaction between this SNV and the shared promoter (FIG. 4B (B)), consistent with the notion that SNV-bound HIF-2α can readily gain access to the promoter for transcriptional activation. Correspondingly, in hypoxic PAEC extracts, oligonucleotides carrying the risk allele SNV rs73184087 (G) displayed preferential and increased binding to HIF-2α, but not HIF-1α, as compared with the major allele (A) (FIG. 4C (C)). Confirming the functionality of such binding, placement of SNV rs73184087 downstream of a luciferase reporter gene demonstrated increased reporter gene expression with the risk (G) allele vs. ancestral (A) allele in the presence of constitutive HIF-2α expression (FIG. 4C (D)). Moreover, in transformed lymphocytes from WSPH Group 1 PAH patients carrying SNV rs73184087 (G/G) vs. (A/A) genotypes, ChIP-qPCR via pulldown of HIF-2α demonstrated a significant enrichment of binding to the (G/G) vs. (A/A) SNV in hypoxia (FIG. 4C (E)). To confirm the long-range interactions of this SNV regardless of its genotype with the shared promoter, using transformed PAH lymphocytes carrying SNV rs73184087 (G/G) or (A/A) genotypes, 3C assays revealed SNV-promoter interaction driven by either the (G/G) or (A/A) genotype (FIG. 4D (F)). A 3C assay using PAECs with the A/A genotype confirmed a specific interaction between the SNV and promoter (FIG. 4D (G)) but not upstream or downstream of the TSS/promoter (FIG. 4D (H)). Finally, under cobalt (II) chloride treatment where HIF-α is stabilized in normoxia (FIG. 4D (I, J)), lymphocytes with (G/G) genotype increased KMT2E-AS1 and KMT2E more robustly as compared to those with (A/A) genotype. Together, these data define an intronic SNV rs73184087 (G) allelespecific mechanism by which HIF-2α controls expression of the IncRNA-KMT2E pair, thus offering a mechanistic explanation underlying the enrichment of SNV rs73184087 (G) allele in WSPH Group 1 PAH.

Mouse IncRNA 5031425E22 phenocopies the endothelial actions of KMT2E-AS1 and depends upon a 500 bp conserved sequence. We wanted to determine if mouse IncRNA E22 carries similar activity as KMT2E-AS1 in PAECs. Specifically, we found that hypoxia upregulates E22 and KMT2E in mouse PAECs, mirroring the regulation of KMT2E-AS1 in human PAECs. Lentiviral forced expression of E22 drove consequent reduction of oxygen consumption and increased glycolysis. As with KMT2E-AS1, this mouse IncRNA controlled similar endothelial pathophenotypes including migration, contraction, and regulation of vasomotor effectors.

Computational predictions of secondary structures of E22 and KMT2E-AS1 revealed putative conserved similarities across these mouse and human isoforms, indicating its importance in this IncRNA's conserved functions. Yet, because of the genomic proximity of this region to the IncRNA-KMT2E promoter, it was possible that the chromosomal region encoding this sequence was also important in controlling canonical promoter function in cis rather than IncRNA function in trans, as reported for other IncRNAs. To clarify these roles, we used a deletion mutant analysis to map a sequence responsible for a IncRNA-dependent function, such as VEGFA induction. This approach identified a 550-600 bp region in the 5′ end of the IncRNA transcript, conserved in both KMT2E-AS1 and mouse E22. This region corresponded to the same functional domain of KMT2E-AS1 that controlled the interaction of H3K4me3 with KMT2E and thus the level of H3K4me3. Finally, by reporter gene assay, deletion of this region did not affect canonical IncRNA-KMT2E promoter activity, emphasizing the importance of this region for IncRNA activity. Therefore, these data demonstrated that mouse E22 and its conserved 550 bp domain can serve as a surrogate to define the in vitro and in vivo causative pathobiology of human KMT2E-AS1.

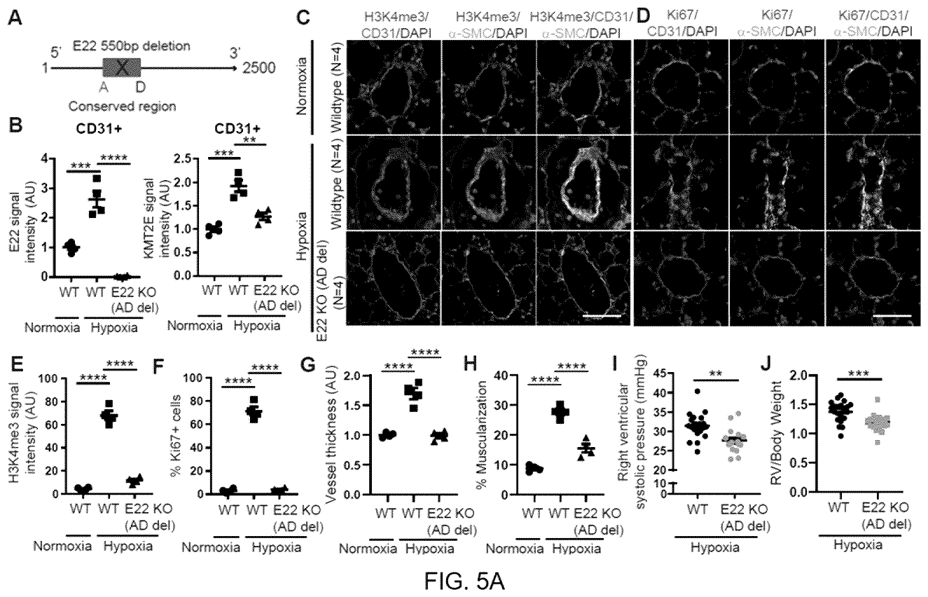

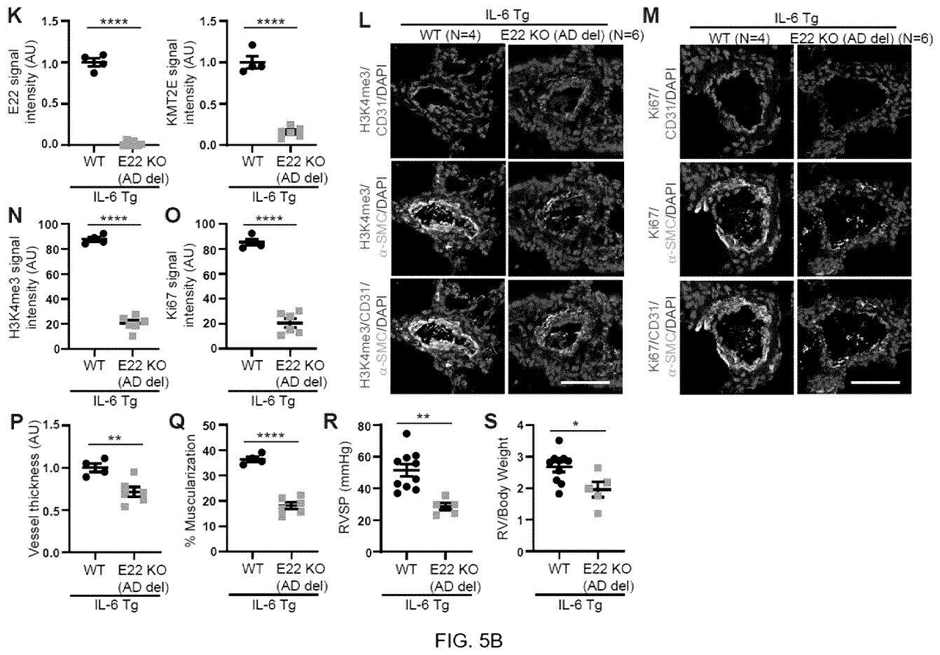

Mice carrying a deletion in the conserved sequence of E22/KMT2E-AS1 are protected from PH in vivo. To determine the direct in vivo pathogenic relevance of the conserved activity of these mice and human IncRNA homologs, we utilized CRISPR/Cas9 technology to generate a mouse genetically deficient specifically in the conserved 550 bp sequence in E22 responsible for control of PAEC activity (FIG. 5A (A)). These knockout mice displayed decreased E22 and KMT2E in CD31-positive pulmonary vascular endothelial cells (FIG. 5A (B)), consistent with knockdown of KMT2E-AS1 in human PAECs. Consequently, under conditions of hypoxic PH, in both lung tissue and CD31-positive PAECs, H3K4me3 was downregulated in knockout mice as compared with wild type littermate controls (FIG. 5A (C E)). This was accompanied by downstream reduction of VEGFA and EDN1, consistent with our studies of cultured PAECs (FIG. 3B (J) and FIG. 3C (Q)) and control of VEGFA by H3K4me3 in hypoxia (FIG. 3B (J)). As in cultured cells, in situ staining of PAECs displayed lower levels of the proliferation marker Ki67 (FIG. 5A (D, F)). Correspondingly, we found that knockout mice were protected from histologic and hemodynamic manifestations of hypoxic PH, including demonstrating decreased pulmonary remodeling/muscularization, right ventricular systolic pressure (RVSP), and right ventricular remodeling (RV to body weight ratio) (FIG. 5A (G-J)) but without other differences in heart rate, blood pressure, or echocardiographic measures of left ventricular (LV) function. To address the possibility of confounding, off-target CRISPR/Cas9 editing, a separate mouse line utilizing alternate guide primer pairing was generated with a smaller conserved sequence deletion (300 bp). These mice exhibited similar reductions of E22 and RVSP.

To model angioproliferative Group 1 PAH, the same mice carrying the 550 bp deletion were crossed with the pulmonary interleukin-6 transgenic (IL-6 Tg) mouse and exposed to chronic hypoxia. We observed decreased E22 and KMT2E (FIG. 5K; FIG. S5A), H3K4me3 (FIG. 5A (L) and FIG. 5B (N)), VEGFA and EDN1, and Ki67 (FIG. 5A (M) and FIG. 5B (O), in CD31-positive pulmonary vascular endothelial cells. This was accompanied by a reduction of vascular remodeling, RVSP, and RV/body weight ratio (FIG. 5B (P-S)) but no differences in heart rate, blood pressure, or LV function. Together, these findings demonstrate that this IncRNA is necessary in promoting experimental Group 1 and 3 PH in vivo, via epigenetic control of endothelial proliferation.

### Example 2—Preparation of iPSCs Homozygous for G at rs73184087

Lymphoblastoid cell lines (LCLs) carrying homozygous G or A at rs73184087 were cultured in RPMI 1640 (Gibco) supplemented with 15% fetal bovine serum (FBS), 1% MEM nonessential amino acids, 1 mM sodium pyruvate, and 10 mM HEPES buffer at 37° C. and 5% CO₂ in a humidified incubator. The LCLs were electroporated with the Neon™ Transfection System 10 μL Kit (MPK1096; Thermo Fisher Scientific) using 1.0 μg of each plasmid (pCXLE-hOCT3/4-shp53, pCXLE-hSK and pCXLE-hUL, Addgene) expressing OCT4, SOX2, KLF4, I-MYC, LIN28 and p53 shRNAs, according to the manufacturer's instructions. The transfected LCLs were transferred to a 12-well plate and incubated for 24 h. At 24 h after electroporation, cells were transferred to a matrigel-coated 12-well plate and supplemented with iPSC reprogramming medium TeSR-E7 (Stemcell Technologies). When iPSC colonies started to appear, cells were then cultured in mTeSR1 media (Stemcell Technologies) and maintained in a hypoxic incubator (5% O₂). Colonies were manually picked for further expansion. See, e.g., Fujimori K, et al. Modeling neurological diseases with induced pluripotent cells reprogrammed from immortalized lymphoblastoid cell lines. Mol Brain. 2016 Oct. 3;9(1):88 describing examples of use of patient-specific iPSCS for modeling of diseases.

### Example 3—Substitution of G at rs73184087

In light of the data presented herein and in U.S. Provisional Patent Application No. 63/334,377, indicating the direct risk and functionality related to the presence of a G at rs73184087, the G of one or both alleles of a patient or cell may be substituted with another nucleobase, such as an A, a T, or a C, by gene editing techniques. In vitro, ex vivo, or in vivo editing may be accomplished by known techniques as would be apparent to a person of ordinary skill in the genetics and medical arts (see, e.g., Nishida K, et al. Science. 2016 Sep. 16;353(6305):aaf8729; Komor A C, et al. Nature. 2016 May 19;533(7603):420-4; and Gaudelli N M, et al. Nature. 2017 Nov. 23;551(7681):464-471). CRISPR/CAS9 editing or base editing may be implemented according to standard protocols, using, for example and without limitation, guide RNAs (gRNAs) provided in Table 1.

TABLE 1
Exemplary gRNAs for substituting the G at rs73184087
to an A, a T, or a C.

| | | | | |
|---|---|---|---|---|
| | | | On- | Off- |
| Sequence (5′→3′) | | | target | target |
| [SEQ ID NO:] | PAM | Cutting Locus | Score | score |

1: for CRISPR/Cas9 editing (A>G, C, T)

| | | | | |
|---|---|---|---|---|
| TTAAAAATATATAGAATAAG [1] | AGG | chr7:−105087906 | 48.5 | 33.8 |
| ATGTTCATTATGTTTTCTCT [2] | TGG | chr7:+105087977 | 44.6 | 37.9 |
| AAAGGGATACTAAAGGAAAA [3] | GGG | chr7:−105087995 | 47.6 | 34.4 |
| AGAATATATAAAGAACTTCT [4] | GGG | chr7:−105084140 | 42.8 | 45.5 |

2: For Base editing (C>T, G>A on complementars trand)

| | | |
|---|---|---|
| | | Base edit |
| ATATATAGAATAAGAGGAAC [6] | TGA | C6>T6 |

### Example 3—Prime Editing

In one example, prime editing, e.g. as described in U.S. Pat. No. 11,447,770 B1, and related publications (see also, International Patent Publication No. WO2020191242 A1 and Anzalone A V, et al. Search-and-replace genome editing without double-strand breaks or donor DNA, Nature. 2019 December;576(7785):149-157, as well as Chen P J, et al. Cell. 2021 Oct. 28;184(22):5635-5652.e29 Nelson J W, et al. Nat Biotechnol. 2022 March;40(3):402-410, among many others). A PE2-type prime editing process may be used, including pegRNA based on the sequence of FIG. 1B, including a single-guide RNA (PE2 guide RNA), the primer binding site (PBS), and the reverse transcription template (RTT). Suitable pegRNA sequences can readily be determined by a person of ordinary skill, for example using software tools including, without limitation: PrimeDesign (github.com/pinellolab/PrimeDesign, Hsu JY , et al. Nat Commun. 2021 Feb. 15;12(1):1034), Easy Prime (https://github.com/YichaoOU/easy_prime, Li Y, et al. Easy-Prime: a machine learning-based prime editor design tool. Genome Biol. 2021 Aug. 19;22(1):235), or PE-Designer (www.rgenome.net/pe-designer/), among others. The pegRNA sequence can then be inserted after a U6 promoter in a plasmid vector (e.g., as commercially-available from Addgene). A plasmid vector for expression of the PE2 Cas9 nickase fused with a reverse transcriptase (Cas9-RT fusion protein) is also provided (e.g., as commercially-available from Addgene). DNA or RNA encoding the pegRNA and the Cas9-RT fusion protein, or pegRNA and Cas9-RT fusion protein-encoding mRNA produced by the plasmid(s), may be delivered to cells of a patient, or stem cells, such as iPSCs by, e.g., standard transfection, transduction, nucleofection protocols and reagents, e.g. as LNPs or virus particles, such as recombinant AAV, Ad, or lentiviral particles. LNPs or viral particles may be delivered to vascular endothelial tissue or a patient's airway, or other sites in a patient by effective delivery routes and methods, such as parenteral, inhaled, topical, or intrathecal routes.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method of increasing histone methylation by KMT2E, such as reducing H3K4 methylation in a patient having one or more Gs at rs73184087, comprising deleting the one or more of the Gs at rs73184087 or substituting the one or more Gs at rs73184087 with A, T, or C in a cell, tissue, or organ of the patient.

2. The method of claim 1 for of treating a condition in a patient having one or more Gs at rs73184087 in which expression of HIF-2α is elevated above normal.

3. The method of claim 1, wherein the patient has a hypoxia-induced condition or hypoxic tissue in the patient.

4. The method of claim 1, wherein the patient is homozygous for G at rs73184087, the method comprising substituting both Gs with A, T, or C in a vascular endothelial cell of the patient.

5. The method of claim 1, wherein the patient has one of pulmonary hypertension, pulmonary arterial hypertension, myocardial infarct, ischemia/reperfusion injury, ischemia, valvular heart disease, congestive heart failure, stroke, cancer, neurodegeneration, thrombus, embolism, and disease-related ischemia.

6. The method of claim 5, wherein the condition is a myocardial infarct, embolism, or thrombus.

7. The method of claim 5, wherein the condition is pulmonary hypertension or pulmonary arterial hypertension.

8. The method of claim 5, wherein the condition is Von Hippel Lindau disease.

9. The method of any one of claims 1-9, wherein the one or more Gs are substituted with A, T, or C using CRISPR/CAS9 editing.

10. The method of claim 9, wherein the CRISPR/CAS9 editing is performed using a guide RNA (gRNA) target sequence selected from: TTAAAAATATATAGAATAAG (SEQ ID NO: 1) the protospacer adjacent motif (PAM) is AGG; ATGTTCATTATGTTTTCTCT (SEQ ID NO: 2) where the PAM is TGG; AAAGGGATACTAAAGGAAAA (SEQ ID NO: 3) where the PAM is GGG; or AGAATATATAAAGAACTTCT (SEQ ID NO: 4) where the PAM is GGG.

11. The method of claim 1, wherein the one or more Gs are substituted with A, T, or C using DNA base editing.

12. The method of claim 1, wherein the one or more Gs are substituted with A, T, or C using prime editing.

13. The method of claim 1, wherein the one or more Gs at rs73184087 are substituted with A.

14. A method of increasing histone methylation by KMT2E, such as increasing H3K4 methylation in a patient having one or more As, Ts, or Cs at rs73184087 comprising substituting at least one of the one or more bases selected from A, T, or C at rs73184087 with G in a cell, tissue, or organ of the patient using gene editing.

15. The method of claim 14, for treating a condition in a patient having one or more bases selected from A, T, or C at rs73184087 in which expression of HIF-2α is reduced below normal, comprising substituting at least one of the one or more bases selected from A, T, or C at rs73184087 with G in a cell, tissue, or organ of the patient using gene editing.

16. The method of claim 14, wherein the patient has an anemia, such as anemia in chronic kidney disease; or peripheral vascular disease and limb ischemia, to increase blood supply and angiogenesis; or is in need of ischemic preconditioning and remote ischemic preconditioning.

17. The method of claim 14, wherein the gene editing is a CRISPR-Cas9 editing, base editing, or prime editing method.

18. An iPSC homozygous for Gs at rs73184087.

19. A cell, such as an endothelial cell, differentiated from the iPSC of claim 18.

20. A method of screening for compounds able to suppress stabilization of KMT2E protein by KMT2E-AS1, comprising culturing under hypoxic conditions a candidate compound with a cell of claim 18 and determining if expression of KMT2E-AS1 is decreased, for example by reduced lysine trimethylation on histone 3 by reduced activity of H3K4me3 or by direct measurement of KMT2E-AS1 detected, for example by quantitative RT-PCR.
